(19) United States
(12) Patent Application Publication
(10) Pub. No.: US 2014/0276136 A1
(43) Pub. Date: Sep. 18, 2014

(54) PERIOPERATIVE FEEDBACK IN ENDOVASCULAR ANEURYSM REPAIR USING PHYSIOLOGICAL MEASUREMENTS

(71) Applicant: VOLCANO CORPORATION, San Diego, CA (US)

(72) Inventors: Neil Hattangadi, San Diego, CA (US); David Goodman, Carlsbad, CA (US)

(73) Assignee: VOLCANO CORPORATION, San Diego, CA (US)

(21) Appl. No.: 14/205,934

(22) Filed: Mar. 12, 2014

Related U.S. Application Data
(60) Provisional application No. 61/792,357, filed on Mar. 15, 2013.

(51) Int. Cl. A61B 5/00; A61B 5/027; A61F 2/07; A61B 5/0215

(52) U.S. Cl. CPC A61B 5/4851; A61B 5/0215; A61B 5/027; A61B 5/6852; A61B 5/6851; A61F 2/07
USPC 600/486; 600/481; 600/505

(57) ABSTRACT

The present invention generally relates to methods for detecting endoleaks associated with EVAR procedures using physiological measurements. The method can involve the taking of functional measurement data in the vicinity of a stent-graft delivered as part of the EVAR procedure and determining the presence of an endoleak based on such data.

### RELATED APPLICATION

This application claims the benefit of and priority to U.S. Provisional No. 61/792,357, filed Mar. 15, 2013, which is incorporated by reference in its entirety.

### FIELD OF THE INVENTION

The present invention generally relates to methods for detecting endoleaks associated with endovascular aneurysm repair using functional parameters.

### BACKGROUND

An abdominal aortic aneurysm (AAA) is an abnormal swelling of the lower part of the aorta that extends through the abdominal area. The aorta is the primary blood vessel that transports blood from the heart to the rest of the body. The walls of aorta are elastic, which allow the vessel to be filled with blood under high pressure. An aneurysm occurs when the arterial walls become weakened and distended. Many factors can contribute to the weakening of arterial walls, including atherosclerosis, high cholesterol, hypertension, and smoking.

An aneurysm that has become too large may rupture, which is extremely dangerous. Symptoms of a ruptured aneurysm include excruciating pain of the lower back, flank, abdomen and groin. Bleeding associated with the rupture often leads to hypovolemic shock, and if left untreated, will result in a relatively quick death.

Conventional methods of repairing abdominal aortic aneurysms include endovascular aneurysm repair or EVAR. In the EVAR procedure, a stent graft is inserted into the aneurysm through small incisions in the groin. The stent-graft reinforces the weakened part of the vessel from the inside and creates a new channel through which the blood flows, eliminating the risk of rupture. A primary concern associated with EVAR is that, despite placement of the stent-graft, blood may continue to flow into the aneurysm, in what is commonly known as an endoleak. Endoleaks arising after grafting may be attributed to an incomplete sealing between the stent-graft and the aortic wall or defects within the stent-graft itself. Endoleaks are the major cause of complications in EVAR procedures, and thus failure in endoluminal treatment of AAA. When an endoleak occurs, it causes continued pressurization of the aneurysm sac and may leave the patient at risk of an AAA rupture and subsequently, immediate death.

### SUMMARY

The present invention provides a method for detecting endoleaks using functional parameters, such as flow or pressure. For example, a pressure sensing guidewire can be maneuvered to site where the stent-graft was placed. Once positioned, the pressure wire can collect the appropriate data, which can then be used to discern the presence of endoleaks. For instance, a decrease in pressure in the vicinity of the stent-graft relative to the pressure further away from the graft may indicate the presence of an endoleak. If no drop in pressure is detected, the stent-graft has effectively treated the aneurysm without generating endoleaks. As contemplated by the invention, any difference between the functional measurements is indicative of endoleaks.

Any functional measurement can be used to discern the presence of endoleaks. Functional measurements can include, for example, determinations of pressure and/or flow in the vicinity of the stent graft and at a point away from the stent-graft, which can then be compared to discern the presence of endoleaks. Other suitable functional measurements can involve manipulations of the pressure and/or flow data to arrive at other functional parameters, including without limitation, fractional flow reserve (FFR), instantaneous wave-free ratio (iFR), and coronary flow reserve (CFR).

The collection of functional measurements typically involves inserting a pressure, flow, or combination wire into the vessel to take the functional measurement. Any pressure, flow, or combination wire can be used in accordance with the invention. Exemplary functional measurement devices suitable for use in practicing the invention include FloWire Doppler Guidewire and the ComboWire XT Guidewire by Volcano Corporation. The guidewire can then be used to measure a functional parameter distal of the stent-graft and at a point more proximal to the stent-graft. A difference in the functional parameter (an increase or decrease in the functional parameter, depending on the particular parameter) is indicative of an endoleak.

Methods of the invention are useful in verifying the effectiveness of the EVAR procedure. Exclusion of the aneurysm sac is the main goal of the stent-graft treatment, and clinical success is defined by the “total exclusion” of the aneurysm. By confirming the absence of endoleaks using the provided methods, the aneurysm can be deemed to have been totally excluded. In addition, the early identification of endoleaks at the time of surgery (perioperatively) can avoid complications at a later time and increase patient mortality.

### BRIEF DESCRIPTION OF THE DRAWINGS

FIG. 1 illustrates a guidewire system for obtaining functional measurements.

FIG. 2 depicts a guidewire of the guidewire system of FIG. 1.

FIG. 3 depicts a distal portion of a guidewire with functional measurement sensors.

FIG. 4 depicts a cross-sectional view of the distal portion of the guidewire shown in FIG. 3.

FIGS. 5-6 illustrate methods of detecting endoleaks of the invention according to certain aspects.

### DETAILED DESCRIPTION

The present invention provides methods for detecting endoleaks through the use of functional or physiological parameters. The method can involve taking a first functional measurement at a point relatively distal to a stent-graft delivered as part of an EVAR procedure and taking a second functional measurement at a point relatively proximal to the stent-graft, and comparing the first and second functional measurements. A difference between the first and second functional measurements is indicative of an endoleak. In the same manner, methods of the invention can also be used to monitor endotension. Endotension is defined as pressure within the aneurysm sac without evidence of endoleak as the cause.

Any functional or physiological measurement is useful for practicing the invention. Exemplary physiological parameters include blood pressure or flow (velocity) inside the abdominal aorta within the vicinity of the stent-graft. In certain aspects of the invention, these initial functional measurements may be further processed to determine other clinically relevant measurements, such as Fractional Flow reserve measurements, Coronary Flow reserve measurements, instantaneous wave-free ratio (iFR), combined P-V curves.

Coronary flow reserve is defined as the ratio of maximal coronary flow with hyperemia to normal flow. Coronary flow reserve signifies the ability of the myocardium to increase blood flow in response to maximal exercise. A ratio at or above 2 is considered normal. Coronary flow reserve measures the velocity of the flow. Fractional flow reserve measure pressure differences across a portion of a vessel to determine whether a level of constriction or stenosis of the vessel will impede oxygen delivery to the heart muscle. Specifically, Fractional flow reserve is a ratio of a level of pressure distal to a portion of a vessel under examination to a level of pressure proximal to a portion of a vessel under examination. When used in methods of the invention, changes in coronary flow reserve or fractional flow reserve within a vessel having an aneurysm treatment are indicative of an endoleak.

P-V loops provide a framework for understanding cardiac mechanics. Such loops can be generated by real time measurement of pressure and volume within the left ventricle. Several physiologically relevant hemodynamic parameters such as stroke volume, cardiac output, ejection fraction, myocardial contractility, etc. can be determined from these loops.

To generate a P-V loop for the left ventricle, the LV pressure is plotted against LV volume at multiple time points during a single cardiac cycle. The presence of an endoleak can alter the curve/shape of P-V loop from a normal P-V loop.

The instantaneous wave-free ratio (iFR) is a vasodilator-free pressure-only measure of the hemodynamic severity of a coronary stenosis comparable to fractional flow reserve (FFR) in diagnostic categorization.

It has been shown that distal pressure and velocity measurements, particularly regarding the pressure drop-velocity relationship such as Fractional Flow reserve (FFR), Coronary flow reserve (CFR), iFR, and combined P-V curves, reveal information about the aneurysm treatment. As contemplated by the invention, these parameters can also be used in the detection of endoleaks. For example, in use, a functional flow device may be advanced to a location relatively distal to an implanted stent-graft. The pressure and/or flow velocity may then be measured for a first time. Then, the device may be advanced to a location relatively proximal to the stent-graft and the pressure and/or flow is measured for a second time. The pressure and flow relationships at these two time points are then compared to assess the presence of endoleaks and provide improved guidance for any coronary interventions. The ability to take the pressure and flow measurements at the same location and same time with a combined pressure/flow guidewire, improves the accuracy of these pressure-velocity loops and therefore improves the accuracy of the diagnostic information.

Coronary flow reserve, Fractional flow reserve, iFR, and P-V loops may require measurements taken at different locations in the artery. In order to provide measurements for these parameters, systems and methods of the invention may assess pressure and flow at a first location of the data collector against a second location of the data collector within the vasculature. For example, a first location that is distal to a segment of a vessel under examination and a second location that is proximal to that segment of a vessel.

In order to obtain the physiological data described above, methods of the invention may involve the use of a functional measurement device. The functional measurement device may be equipped with a pressure sensor, a flow sensor, or any combination thereof. Exemplary functional measurement devices suitable for use in practicing the invention include FloWire Doppler Guidewire and the ComboWire XT Guidewire by Volcano Corporation.

In particular embodiments, a pressure sensor can be mounted on the distal portion of a flexible elongate member. In certain embodiments, the pressure sensor is positioned distal to the compressible and bendable coil segment of the elongate member. This allows the pressure sensor to move along with the along coil segment as bended and away from the longitudinal axis. The pressure sensor can be formed of a crystal semiconductor material having a recess therein and forming a diaphragm bordered by a rim. A reinforcing member is bonded to the crystal and reinforces the rim of the crystal and has a cavity therein underlying the diaphragm and exposed to the diaphragm. A resistor having opposite ends is carried by the crystal and has a portion thereof overlying a portion of the diaphragm. Electrical conductor wires can be connected to opposite ends of the resistor and extend within the flexible elongate member to the proximal portion of the flexible elongate member. Additional details of suitable pressure sensors that may be used with devices of the invention are described in U.S. Pat. No. 6,106,476. U.S. Pat. No. 6,106,476 also describes suitable methods for mounting the pressure sensor 104 within a sensor housing.

A flow sensor can be used to measure blood flow velocity within the vessel, which can be used to assess coronary flow reserve (CFR). The flow sensor can be, for example, an ultrasound transducer, a Doppler flow sensor or any other suitable flow sensor, disposed at or in close proximity to the distal tip of the guidewire. The ultrasound transducer may be any suitable transducer, and may be mounted in the distal end using any conventional method, including the manner described in U.S. Pat. Nos. 5,125,137, 6,551,250 and 5,873,835.

A pressure sensor allows one to obtain pressure measurements within a body lumen. A particular benefit of pressure sensors is that pressure sensors allow one to measure of FFR in vessel. FFR is a comparison of the pressure within a vessel at positions prior to the stenosis and after the stenosis. The level of FFR determines the significance of the stenosis, which allows physicians to more accurately identify clinically relevant stenosis. For example, an FFR measurement above 0.80 indicates normal coronary blood flow and a non-significant stenosis. Another benefit is that a physician can measure the pressure before and after an intraluminal intervention procedure to determine the impact of the procedure.

The acquisition of functional measurements typically involves the insertion of a pressure, flow, or combination guidewire into a blood vessel and measuring pressure and/or flow inside the vessel with the device. In practice, measuring pressure and/or flow inside the vessel may also involve injecting a local anesthetic into the skin to numb the area of the patient prior to surgery. A puncture is then made with a needle in either the femoral artery of the groin or the radial artery in the wrist before the provided guidewire is inserted into the arterial puncture. Once positioned, the guidewire may then be used to measure pressure and/or flow in the vessel.

Once the device is inside the vessel, the effectiveness of the EVAR procedure can be verified and the presence of endoleaks detected through the assessment of functional data. As discussed above, the sensing device can be maneuvered to a position relatively distal of the implanted stent-graft. At this position, a first functional measurement is taken. The sensing device is then advanced to a position relatively proximal to the implanted stent-graft and a second functional measurement is taken. The appropriate distance away from the implanted stent-graft can be determined empirically and is within the ordinary skill of the art. In addition, the distal and proximal measurement positions are still within a limited space, i.e., the abdominal aorta, facilitating the identification of the positions. The two measurements can then be compared with a difference between the first and second measurements being indicative of an endoleak. For example, a drop in pressure near the vicinity of the stent-graft may indicate the presence of an endoleak. Because there is still a hole in the vessel wall after the EVAR procedure, the abdominal aorta is unable to maintain pressure in the vicinity of the stent-graft. In addition, an increase in flow may also indicate the presence of an endoleak. This is because blood is still flowing out the ineffectual seal made by the stent-graft, which can be detected as an increase in flow.

Reference will now be made to endovascular aneurysm repair (EVAR) procedure. Methods of the invention are useful with all EVAR related procedures, including without limitation, EVAR, hybrid EVAR, Common Iliac Artery EVAR, and Thoracic EVAR (TEVAR). EVAR is typically conducted in a sterile environment, usually a theatre, under x-ray fluoroscopic guidance. The patient is usually administered an anesthetic prior to conducting the procedure. A puncture is then made with a needle in the femoral artery of the groin. An introducer or vascular sheath is then inserted into the artery with a large needle, and after the needle is removed, the introducer provides access for guidewires, catheters, and other endovascular tools, such as the stent-graft used to treat the abdominal aneurysm.

Diagnostic angiography images or ‘runs’ are captured of the aorta to determine the location on the patient's renal arteries, so the stent graft can be deployed without blocking them. Blockage may result in renal failure, thus the precision and control of the graft stent deployment is extremely important. The main ‘body’ of the stent graft is placed first, follow by the ‘limbs’ which join on to the main body and sit on the Aortic Bifurcation for better support, and extend to the Iliac arteries. The stent graft (covered stent), once positioned, serves as an artificial lumen for blood to flow down, and not into the surrounding aneurysm sac. Accordingly, pressure is taken off the aneurysm wall, which itself will thrombose in time.

For certain occasions that the aneurysm extends down to the Common Iliac Arteries, a specially designed graft stent, named as Iliac Branch Device (IBD), can be used, instead of blocking the Internal Iliac Arteries, but to preserve them. The preservation of the Internal Iliac Arteries is important to prevent Buttock Claudication, and to preserve the full genital function.

A variation of EVAR is the Hybrid Procedure. A hybrid procedure occurs in the angiography room and aims to combine endovascular procedures with limited open surgery. In this procedure the stent graft deployment is planned to combine with an open operation to revascularise selected arteries that will be “covered” by the stent graft i.e. deprived of arterial inflow. In this method more extensive EVAR devices can be deployed to treat the primary lesion while preserving arterial flow to critical arteries.

Thoraco-abdominal aneurysms (TAA) typically involve such vessels and deployment of the EVAR device will cover important arteries e.g. visceral or renal arteries, resulting in end organ ischaemia which may not be survivable. The open operation component aims to bring a bypass graft from an artery outside the stent graft coverage to vital arteries within the coverage region. This component adds to the EVAR procedure in time and risk but is usually judged to be lesser that the risk of the major totally open operation.

The above procedures aim to reduce the morbidity and mortality of treating certain types of arterial disease. The occurrence of endoleaks, however, can significantly increase the risk associated with EVAR procedures. An endoleak is characterised by persistent blood flow within the aneurysm sac following endovascular aneurysm repair. Normally the aortic stent-graft used for EVAR excludes the aneurysm from the circulation by providing a conduit for blood to bypass the sac. An improperly positioned or defective graft, however, can result in an ineffectual seal and result in the formation of endoleaks.

An endoleak is a common complication of EVAR and is found in a significant number of patients intraoperatively (seen on the on-table angiogram after stent deployment), as well as during follow-up. This somewhat common occurrence greatly reduces the overall effectiveness of the EVAR procedure. Although some endoleaks appear to be unavoidable due to the presence of pre-existing patent branch vessels arising from the aneurysm sac, others occur as a result of poor patient/graft selection.

In either situation, there is an immediate need to monitor the occurrence of endoleaks, preferably during the procedure itself (perioperatively). Methods of the invention address this need and can be used perioperatively. While the patient is still “open” and has the introducer used for delivering the stent-graft still inside him, the same introducer can be used to manuever the functional measurement guidewire to the site of the implanted graft and acquire functional data (pressure and/or flow measurements, for example) near the site of implantation.

Endoleaks are often asymptomatic, however as flow within the aneurysm sac is at systemic or near-systemic pressure, if untreated, the aneurysm may expand and is at risk of rupture. As such aneurysm expansion following EVAR always warrants investigation for endoleak.

Endoleaks are typically classified as either type I, type II, type III, type IV, and type VI endoleaks.

Type I endoleaks occur as a result of an inadequate seal at the site of the graft attachment. It may occur at the proximally end, distal end or where the components overlap. Blood flow leaks alongside the graft into the aneurysm sac. They are often the result of unsuitable patient (aneurysm) selection or device selection, but can also occur if the graft migrates. Type I leaks are always considered significant as they do not tend to resolve spontaneously.

Type II endoleaks are the most common. In this situation, retrograde flow though branch vessels continues to fill the aneurysm sac. The most common culprit vessels are lumbar arteries, inferior mesenteric artery or internal iliac artery. This type of leak has been a substantial number of cases. It usually resolves spontaneously over time and requires no treatment. Embolisation of the branch vessel is indicated if the aneurysm sac continues to expand in size.

Type III endoleaks are caused by mechanical failure of the stent-graft. There may be a fracture of the stent-graft, hole or defect on the graft fabric, or junctional separation of the modular components. Causes may relate to defective device material, extreme angulation of a segment predisposing to fracture, or improper overlap of the modular components during insertion.

Type IV endoleaks occur when blood leaks across the graft due to its porosity. It does not require any treatment and typically resolves within a few days of graft placement.

Type V “leak” (also referred to as endotension) is not a true leak but is defined as continued expansion of the aneurysm sac without evidence of a leak site. It is also referred to as endotension. Its origin is still unclear but is believed to be due to pulsation of the graft wall with transmission of the pulse wave through the perigraft space (aneurysm sac) to the native aneurysm wall.

Methods of the invention can further encompass treatment of the endoleak upon detection based on functional parameters. Treatment will depend on the type of endoleak.

Type I leaks (above, below of between graft components) are generally treated as soon as detected. Extension cuffs or covered stents can be inserted at the leaking graft end to improve the seal, or embolisation of the leak site with glue or coils can be used. Rarely, if detected intra-operatively during EVAR, conversion to an open procedure may be required if endovascular methods of sealing the leak are unsuccessful.

Type II leaks (retrograde flow through branch) usually spontaneously thrombose. As such at many institutions these leaks are not treated immediately; watchful waiting is employed and if the leak persists it is treated by embolising the branch vessel with glue or coils. Pre-emptive embolisation of potential sources of collateral flow is sometimes performed prior to stent-graft insertion, particularly the internal iliac artery in select cases. Pre-emptive embolisation of other branch vessels is controversial.

Type III leaks (graft mechanical failure) do not spontaneously resolve and are therefore treated immediately, usually with additional stent-graft components.

Type IV leaks (graft porosity) require no treatment.

Type V leaks (endotension) are controversial but when continued growth of the aneurysm sack is demonstrated further treatment with additional endoluminal components (cuffs or extensions) may be successful. Alternatively, conversion to an open repair may be necessary.

The above described methods of the invention can be performed with a functional measurement catheter or guidewire. Preferred devices include a pressure sensor, flow sensor, or combination thereof. The following sets forth an exemplary functional measurement guidewire. It is understood that the below guidewire and/or its sensors could be adapted into catheters.

Referring now to FIG. 1, FIG. 1 provides a schematic illustration of a functional measurement guidewire being used during a procedure to assess endoleaks and other aneurysm leaks in a patient 22. The patient 22 is shown lying on a bed 23 in a surgical lab. The guide wire 21 is used with apparatus 24 which consists of a cable 26 which connects the guide wire 21 to an interface box 27 as shown or directly to a connected to an instrument. such as a computing device (e.g. a laptop, desktop, or tablet computer) or a physiology monitor. Interface box 27 is connected by another cable 28 to a control console 29 which has incorporated as a part thereof a video screen 31 on which a waveform 32 displaying functional measurements may be provided. For example, the ECG measurements may appear as traces 32, 33 and 34.

The guide wire 21 is shown more in detail in FIG. 2 and as shown therein, the guide wire 21 can be constructed utilizing the various constructions as shown in U.S. Pat. Nos. 5,125,137; 5,163,445; 5,174,295; 5,178,159; 5,226,421; and 5,240,437. As disclosed therein, such a guide wire consists of a flexible elongate element 41 having a proximal and distal extremities 42 and 43 and which can be formed of a suitable material such as stainless steel having an outside diameter for example of 0.018″ or less and having a suitable wall thickness as for example, 0.001″ to 0.002″ and conventionally called a “hypotube” having a length of 150-170 centimeters. Where a smaller guide wire is desired, the hypotube 41 can have an exterior diameter of 0.014″ or less. Typically such a guide wire includes a core wire (not shown) of the type disclosed in the above identified patents which extends from the proximal extremity to the distal extremity of the flexible elongate element 41 to provide the desired torsional properties for guide wires (See U.S. Pat. No. 5,163,445, col. 18:40-51) to facilitate steering of the guide wire 21 in the vessel.

A coil spring 46 is provided and is formed of a suitable material such as stainless steel. It has an outside diameter of 0.018″ and is formed from a wire having a diameter of 0.003″. The spring 46 is provided with a proximal extremity 47 which is threaded onto the distal extremity 43 of the flexible elongate member 41. The distal extremity 48 of the coil spring 46 is threaded onto the proximal extremity 49 of an intermediate or transition housing 51 such as disclosed in U.S. Pat. No. 5,174,295, formed of a suitable material such as stainless steel having an outside diameter of 0.018″ and having a suitable wall thickness as for example, 0.001″ to 0.002″. The housing includes one or more sensors, such as pressure sensor or flow sensor (See FIG. 3).

A torquer 66 of the type described in U.S. Pat. No. 5,178,159 is mounted on the proximal extremity 42 of the flexible elongate member 41 for causing a rotation of a guide wire 21 when used in connection with catheterization procedures in a manner well known to those skilled in the art.

The proximal extremity 42 is also provided with a plurality of conducting sleeves (not shown) of the type disclosed in U.S. Pat. No. 5,178,159. In the present invention, one or more additional sleeves can be provided to make connection to the conductors hereinafter described. The proximal extremity 42 of the flexible elongate member is removably disposed within a housing 68 of the type described in U.S. Pat. Nos. 5,178,159, 5,348,481 and 5,358,409 that makes electrical contact with the sleeves on the proximal extremity 42 while permitting rotation of the sleeves and the flexible elongate member 41. The housing 68 carries female receptacles (not shown) which receive the sleeves and which are connected to a cable 71 connected to a connector 72. The connector 72 is connected to another mating connector 73 carried by the cable 26 and connected into the interface box 27.

In addition, FIG. 3 shows a sensor tip 400 of a guidewire 21 that may be suitable to use with methods of the invention. The combination sensor tip 400 includes a pressure sensor 404 within sensor housing 403, and optionally includes a radiopaque tip coil 405 distal to proximal coil 406.

FIG. 4 gives a cross-sectional view through combination sensor tip 400, showing ultrasound transducer 501 disposed therein. The ultrasound transducer 501 may be any suitable transducer, and may be mounted in the distal end using any conventional method, including the manner described in U.S. Pat. No. 5,125,137, which is fully incorporated herein by reference. Conductors (not shown) may be secured to the front and rear sides of the ultrasound transducer 501, and the conductors may extend interiorly to the proximal extremity of a guide wire.

The combination sensor tip 400 also includes a pressure sensor 404 also disposed at or in close proximity to the distal end 202 of the combination sensor tip 400. The pressure sensor 404 may be of the type described in U.S. Pat. No. 6,106,476, which is fully incorporated herein by reference. For example, the pressure sensor 404 may be comprised of a crystal semiconductor material having a recess therein and forming a diaphragm bordered by a rim. A reinforcing member may be bonded to the crystal to reinforce the rim of the crystal, and may have a cavity therein underlying the diaphragm and exposed to the diaphragm. A resistor having opposite ends may be carried by the crystal and may have a portion thereof overlying a portion of the diaphragm. Leads may be connected to opposite ends of the resistor and extend proximally within the guide wire. Additional details of suitable pressure sensors that may be used as the pressure sensor 404 are described in U.S. Pat. No. 6,106,476. U.S. Pat. No. 6,106,476 also describes suitable methods for mounting the pressure sensor 404 within the combination sensor tip 400. In one embodiment, the pressure sensor 404 is oriented in a cantilevered position within a sensor housing 403. For example, the sensor housing 403 preferably includes a lumen surrounded by housing walls. When in a cantilevered position, the pressure sensor 404 projects into the lumen of the sensor housing 403 without contacting the walls of the sensor housing 403.

In FIG. 4, ultrasound transducer 501 is illustrated as disposed near distal end 202. One advantage of the sensor housing 403 is that because the sensor housing 403 encloses both the ultrasound transducer 501 and the pressure sensor 404, the need for two separate housings, i.e., one for an ultrasound transducer and one for a pressure sensor, is eliminated. Accordingly, the use of a common sensor housing 403 for the ultrasound transducer 501 and the pressure sensor 404 makes the combination sensor tip 400 easier to manufacture than current designs.

Additionally, the combination sensor tip 400 of the present invention provides for both the ultrasound transducer 501 and the pressure sensor 404 to be disposed near the distal end of the combination sensor tip 400. The combination sensor tip 400 of the present invention is advantageous because by having both the ultrasound transducer 501 and the pressure sensor 404 near its distal end, the combination sensor tip 400 is capable of being positioned distally beyond the fistula. Additionally, the combination sensor tip 400 of the present invention, unlike the prior art, is also able to take measurements from the ultrasound transducer 501 and the pressure 104 at approximately the same location and approximately the same time, thereby resulting in greater consistency of measurements, greater accuracy of measurements, and greater accuracy of placement within the body. Furthermore, placement of both the ultrasound transducer 501 and the pressure sensor 404 near the distal end of the combination sensor tip 400 increases overall flexibility in a guide wire that incorporates the combination sensor tip 400. For example, a prior art guide wire that includes separate sensors, with the pressure sensor being located substantially proximal from the ultrasound transducer, has a longer relatively rigid area that must be devoted to the pressure and flow sensors, i.e., the distance from the ultrasound transducer to the pressure sensor. The present invention, in contrast, substantially reduces or entirely eliminates the distance between the ultrasound transducer and the pressure sensor, thereby allowing for increased flexibility across this length.

It should be noted that in an alternative embodiment of the combination sensor tip 400 (not shown) both the ultrasound transducer 501 and the pressure sensor 404 may be offset from the distal end of the combination sensor tip 400, such as, e.g., 1.5 cm to 3.0 cm from the distal end, but still located in close proximity to each other relative to prior art designs. Thus, the aforementioned advantages over the prior art design are still achieved.

In an alternative embodiment, the pressure sensor housing includes a tubular member having an opening on the outer wall in communication with the lumen and a tip. The tip is constructed of a solder ball. Alternatively a weld, braze, epoxy or adhesive can be used. The lumen of the housing is counter-bored so that the lumen has a smaller inner diameter at the proximal end of the tubular member. For example, the housing may be constructed in the counter-bore fashion with a 0.010″ inner diameter at the proximal end and a 0.012″ inner diameter at the distal end, with the pressure transducer coaxially housed in the lumen. In addition, a flow sensor may be placed in the sensor tip instead of the weld, braze, epoxy or adhesive to provide a combo sensor tip. The advantage of the counter bore is that the housing is easier to make. The transducer is simply slid into place in the lumen and bonded (adhesive or epoxy) where the sides meet the proximal 0.010″ inner diameter 314. The distal 0.012″ inner diameter allows enough room for the pressure sensitive section of the transducer to be free from any contact with the housing. Because of the counter-bored lumen, there is no ledge that has to be made on the outer wall of the lumen, rather the pressure transducer communicates with the outside via an opening in the outer wall of lumen. Constructions suitable for use with a guidewire of the invention are discussed in U.S. Pub. 2013/0030303 to Ahmed, the contents of which are incorporated by reference.

A radiopaque tip coil 405 may be provided at the proximal end of the combination sensor tip 400. The radiopaque tip coil 405 is coupled to a proximal coil 406, and the proximal coil 406 may be coupled to the elongate tubular member. Another improvement of the present invention over current designs that use separate pressure sensor and ultrasound transducer housings is that the present invention provides a smoother transition from the elongate tubular member to the combination sensor tip 400, i.e., the connection between the radiopaque tip coil 405, the proximal coil 406, and the rest of the guide wire is optimized relative to current designs. Specifically, the transition is smoother and more flexible because of the absence of the housing between the radiopaque tip coil 405 and the proximal coil 406. Current designs generally have a tip coil attached to a pressure sensor housing, which in turn is connected to a proximal coil. The present invention eliminates or greatly reduces the separation between the tip coil and the proximal coil that is required in current devices. Suitable coils for use with the present invention are described in U.S. Pat. No. 6,106,476.

In a preferred embodiment, methods of the invention employ a Doppler guidewire wire sold under the name FLOWIRE by Volcano Corporation, the pressure guidewire sold under the name PRIMEWIRE PRESTIGE by Volcano Corporation, or both. Suitable guidewires are also discussed in U.S. Pat. No. 5,125,137, U.S. Pat. No. 5,163,445, U.S. Pat. No. 5,174,295, U.S. Pat. No. 5,178,159, U.S. Pat. No. 5,226,421, U.S. Pat. No. 5,240,437 and U.S. Pat. No. 6,106,476.

FIGS. 5-6 illustrate using methods of the invention to detect the presence of an endoleak after aneurysm treatment. FIG. 5 shows treatment of an aneurysm 800 formed in a blood vessel 802 with use of a vaso-occlusive device 804. Vaso-occlusive devices 804 are used to clog the aneurysm sac 806, thereby prevent blood flow into the sac 806. Typical vaso-occlusive devices and materials include platinum micro-coils, hog hair, microfibrillar collagen, various polymeric agents, material suspensions, and other space filling materials. FIG. 6 shows treatment of an aneurysm 800 formed in a blood vessel 802 with use of a stenting device 808. Exemplary stenting devices 808 capable of restricting blood flow to an aneurysm include meshes or fenestrated structures which are positioned near the neck (opening) of an aneurysm 800 and restrict the flow of blood thereto. In order to detect endoleaks present either treatment shown in FIGS. 5-6, a guidewire 810 with functional measurements sensors 812 is introduced into the blood vessel 802, and used to take functional measurements along the length of the blood vessel 802. Functional measurements are taken at a first location (5A, 6A) proximal to the aneurysm 800, and functional measurement are taken at a second location (5B, 6B) distal to the aneurysm 800. A difference in the functional measurements obtained by the guidewire 810 is indicative of an endoleak.

In alternative embodiments, the guidewire itself may have a first sensor located a first position, and a second sensor located at a second position. In this embodiment, the guidewire may obtain functional measurements proximal and distal to the aneurysm at the same time without requiring movement of the guidewire. For example, the first sensor may be able to take the proximal measurements, and the second sensor may be able to take the distal measurements,

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

The invention may be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes which come within the meaning and range of equivalency of the claims are therefore intended to be embraced therein.

What is claimed is:

1. A method for detecting leaks associated with aneurysm repair, the method comprising:
inserting a detection device inside a vessel; and
taking a first functional measurement with the detection device at a point relatively distal to an prosthesis delivered in conjunction with an aneurysm repair-based procedure;
taking a second functional measurement with the detection device at a point relatively proximal to the prosthesis; and
comparing the second functional measurement to the first, wherein a difference between the first and second measurements indicates the presence of a leak.

2. The method of claim 1, wherein the detection device is selected from the group consisting of a pressure-sensing catheter, a flow-sensing catheter, and a combination pressure/flow-sensing catheter.

3. The method of claim 1, wherein the detection device is selected from a group consisting of a pressure-sensing guidewire, a flow-sensing guidewire, and a combination pressure/flow-sensing wire.

4. The method of claim 1, wherein the functional measurement is selected from the group consisting of pressure, flow, fractional flow reserve (FFR), coronary flow reserve (CFR), or instantaneous wave-free radio (iFR).

5. The method of claim 1, wherein the endovascular prosthesis comprises a stent-graft.

6. The method of claim 1, wherein the vessel comprises an aortic vessel.

7. The method of claim 6, wherein the vessel is an abdominal aortic vessel.

8. The method of claim 6, wherein the vessel is a thoracic aortic vessel.

9. The method of claim 1, wherein the aneurysm repair-based procedure is selected from standard endovascular aneurysm repair (standard EVAR), thoracic endovascular aneurysm repair (TEVAR), Hybrid EVAR, or Iliac Artery EVAR.

10. The method of claim 1, wherein the difference between the first and second measurements comprises a decrease in pressure.

11. The method of claim 1, wherein the difference between the first and second measurements comprises an increase in flow.

12. The method of claim 1, wherein the leak is an endoleak.
